(19) United States
(12) Patent Application Publication
(10) Pub. No.: US 2022/0176004 A1
(43) Pub. Date: Jun. 9, 2022

(54) NEBULIZER DISINFECTING SYSTEM AND METHOD OF USE

(71) Applicant: The Ritedose Corporation, Columbia, SC (US)

(72) Inventors: Shawn Miller, Columbia, SC (US); Jody Chastain, West Columbia, SC (US)

(21) Appl. No.: 17/542,004

(22) Filed: Dec. 3, 2021

Related U.S. Application Data
(60) Provisional application No. 63/122,161, filed on Dec. 7, 2020.

(51) Int. Cl. A61L 9/20; A61M 11/02

(52) U.S. Cl. CPC A61L 9/20; A61M 11/02; A61L 2209/134; A61L 2209/14; A61L 2209/12

(57) ABSTRACT

Devices and methods for reducing the amount of contagions, such as germs, from the exhaled air stream of a nebulizer are provided. Specifically, a nebulizer is provided with a disinfecting chamber in communication with the exhaled air outfeed of a nebulizer that reduces the amount of contagions exiting the outfeed to the environment. An example of such a disinfecting chamber is one that employs a UVC light source.

### CROSS-REFERENCE TO RELATED APPLICATIONS

This application claims the benefit of U.S. Provisional Patent Application No. 63/122,161, filed on Dec. 7, 2020, the entire disclosures of which are hereby incorporated by reference herein.

### FIELD OF THE INVENTION

The present invention relates generally to nebulizer disinfecting systems and methods of using such systems to disinfect exhaled gas exiting from a nebulizer while being used by a patient taking a breathing treatment.

### BACKGROUND OF THE INVENTION

Medical nebulizers are devices that are commonly used to transport medication into the lungs of patients to treat certain conditions and diseases. In the most common type of nebulization, a gas is passed through a liquid in a way such that some of the liquid is entrained in the gas, forming an aerosol. These nebulizers are designed to suspend the fluid in the gas stream, causing the fluid to become atomized by having small particles of the fluid become suspended in the gas stream. Nebulizers are commonly used in inhalation therapies. First, the patient inhales air into the nebulizer which contains the aerosol and the aerosol-laden air containing the medication is then breathed into the patient's lungs. The patient then exhales air back into the environment. The air can either be pressure assisted or not depending on the status of the patient.

A jet or atomizing nebulizer is a relatively simple device which causes a liquid, typically medicine, to be entrained into a gas for inhalation. A nebulizer includes a cup, typically called a nebulizer cup or medicine cup, which is where the liquid medicine is placed for the breathing treatment. The most basic function of the nebulizer is to transfer this liquid in the cup into air that can be inhaled by the user into their lungs. A jet or atomizing nebulizer forces a pressurized gas, typically compressed air or oxygen, through the liquid, thus turning the liquid into an aerosol. This aerosol is created in the headspace of the medicine cup.

The basic mechanism to deliver this aerosol into the lungs of the patient is to force the patient's breath to travel through the headspace of the medicine cup where the aerosol resides. This will allow the patient to breath in the aerosol when the patient inhales, but will also result in the aerosol being released into the room air when the patient exhales. There are various other components that can be added to nebulizers to help control droplet sizes of the liquid in the aerosol and limit medicine loss when exhaling. Examples of such components are mouthpieces and masks to help the patients more comfortably inhale through the nebulizer, and baffles to help form the mist.

Jet nebulizers typically share a common air inlet and exhaled air outlet, which allows for some of the aerosol to be released into the room air when the patient exhales. Until the present COVID-19 pandemic, the release of exhaled air from the patient has generally not been an issue. As a patient exhales, it is not only medication and air that is released into the room air, but anything else that might be entrained in the patient's breath, such as the COVID-19 virus or any other contagious virus. This risk of spreading the COVID-19 virus through the use of jet nebulizers in hospitals has led to a significant reduction in the use of nebulization, resulting in a possible decline in patient care.

The COVID-19 pandemic introduced the world to a range of mitigation strategies that can be used to slow the spread of a virus. With an airborne virus, such as COVID-19, a focus on exhaled air is most critical, as it can be passed from person to person through normal breathing and talking. Social distancing and mask wearing are the primary methods a population can use to limit the airborne spread of germs from person to person, combined with frequent sanitization of hands and surfaces. As the world focused on limiting the spread of the virus through breathing, health care providers evaluated their systems for weaknesses, and the nebulizer was quickly found to be a potential source of viral spread. The issue with nebulizers is that the user breathes in air from the environment, and exhales back into that same environment. If the patient has a virus, then the virus will be exhaled back into the environment with their breath, creating a source of spread. Because of this weakness, the use of nebulizers for breathing treatments has declined significantly as a result of COVID-19 virus mitigation strategies. While COVID-19 has brought this inherent flaw in the nebulizer design to the forefront, there is no reason to believe that the use of nebulizers will return to normal levels without a method of disinfecting the exhaled gas.

The need exists for a device and method for sanitizing the breath that is exhaled through a nebulizer to reduce or eliminate contagions in the exhaled breath and thus reduce the risk of spreading a virus when using nebulizers. The present invention attempts to fulfill what is an immediate and long-felt need.

The foregoing examples of the related art and limitations related thereto are intended to be illustrative and not exclusive. Other limitations of the related art will become apparent to those skilled in the art upon a reading of the specification and a study of the drawings.

### SUMMARY OF THE INVENTION

Briefly, the present invention is directed, in various embodiments, to devices and methods for sanitizing the breath that is exhaled through a nebulizer, reducing contagions in the exhaled air before it is released into the room air.

One embodiment of the present invention uses UVC light to disinfect the exhaled air before releasing it into the room air.

In another embodiment of the present invention, a method is employed that separates the inhaled air stream from the exhaled air stream which provides a high level of control over the potentially contaminated exhaled air. In particularly, two one-way valves would be used, each allowing air to flow in only one direction. The incoming air would flow into the nebulizer through the inlet valve, and the exhaled air would flow out of the nebulizer through the outlet valve. The exhaled air treatment system would be attached to the outlet valve, ensuring that only exhaled air would be treated.

While use of UVC light is one of the disclosed embodiments, any sanitization method or device currently in use, or developed in the future, could replace the UVC light chamber without altering the present invention and all such potential sanitization techniques are included in the scope of the present invention.

In another embodiment, a nebulizer comprises a nebulizer housing; a patient interface; an ambient air infeed for carrying inhaled air to the nebulizer for entraining a medication for administration to a patient's lungs; an exhaled air outfeed for carrying exhaled air from the lungs through the exhaled air outfeed to be treated or contained where the exhaled air may carry contagions entrained therein and could enter the contagions into the environment if not prevented; and a disinfecting chamber attached to the nebulizer for reducing the amount of contagions entering the environment from the exhaled air outfeed.

In yet another embodiment, a nebulizer comprises a nebulizer housing; a patient interface; an ambient air infeed for carrying inhaled air to the nebulizer for entraining a medication for administration to a patient's lungs; an exhaled air outfeed for carrying exhaled air from the lungs through the exhaled air outfeed to be treated or contained where the exhaled air may carry contagions entrained therein and could enter the contagions into the environment if not prevented; and a device that separates inhaled air from exhaled air.

In certain embodiments, the device that separates inhaled air from exhaled air in this embodiment is a T-splitter. The device may comprise two or more one-way valves. In certain embodiments, the device may be positioned between the patient interface and the nebulizer. In other embodiments, the device may be positioned for communication with the exhaled air outfeed. In additional embodiments, the device may be attached to the exhaled air outfeed.

In yet other embodiments, a device for removably attaching to a nebulizer is provided wherein the nebulizer has nebulizer housing, a patient interface, an ambient air infeed for carrying inhaled air to the nebulizer for entraining a medication for administration to a patient's lungs, an exhaled air outfeed for carrying exhaled air from the lungs through the exhaled air outfeed to be treated or contained where the exhaled air may carry contagions entrained therein and could enter the contagions into the environment if not prevented. The device in this separate embodiment is a T-splitter comprising at least two, but potentially more, one-way valves and may be adapted for positioning between the patient interface and the nebulizer, for communication with the exhaled air outfeed, and for attachment to the exhaled air outfeed.

### BRIEF DESCRIPTION OF THE DRAWINGS

The accompanying drawings are not necessarily drawn to scale, but are provided to illustrate the parts that make up embodiments of the invention. Critical components of the drawings are labeled, to ease references contained in the detailed description. When the same component exists in multiple drawings, a single label is used for that component. Several example embodiments are illustrated in an effort to demonstrate the flexibility of the invention; however, these drawings are not meant to include all potential embodiments of the invention.

FIG. 1 is a schematic illustration of an exemplary jet nebulizer and shows a typical inhaled air and exhaled air pathways.

FIG. 2 is a schematic illustration of the material balance of a jet nebulizer showing the introduction of germs or other contagions into the nebulizer.

FIG. 3 is a schematic illustration of a UVC light chamber connected to the ambient air side of a nebulizer.

FIG. 4 is a schematic illustration of a portion of a nebulizer showing separated ambient air interfaces using valves to split the ambient air infeed from the exhaled air outfeed.

FIG. 5 is a schematic illustration of a portion of a nebulizer with an ambient air T-splitter that separates the standard ambient air interface into an ambient air infeed and an exhaled air outfeed.

### DETAILED DESCRIPTION OF PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

Most people know about different types of medicine, but they do not think about the complications that can arise with delivering the medication to the patient. A simple example is delivering medicine via a pill. Everyone likely knows someone who is unable or struggles to swallow a pill, which creates difficulty in medicating the patient. This difficultly is often ameliorated by breaking the pill into smaller pieces or administering the medication in liquid form. However, the inability to deliver medicine to a patient can create a life-threatening situation.

Breathing issues, such as COPD or asthma, are commonplace and patients with these issues are often administered inhalant-type medications through a metered-dose inhaler. While these types of devices are commonly seen and used, some patients are, however, unable to use a metered-dose inhaler, and require another type of delivery system. The most common alternate delivery system to the metered-dose inhaler is the nebulizer, which serves the same purpose as the metered-dose inhaler to administer the delivery of medicine into the lungs.

Several methods exist for removing germs and bacteria from air, and most of these could be adapted for use in nebulizer exhaust. However, because nebulizer users are typically patients who have difficulty breathing, it is important that the method of scrubbing germs, bacteria, and contagions from the nebulizer exhaust not add back pressure to the exhaust which would make it more difficult for the user to breathe.

There are several types of UV light, and they are classified by how much energy they have. UVA is the lowest energy and safest type of UV light, and this is what people are mostly exposed to while in the sun. A small portion of sunlight contains a higher energy and more dangerous type of UV, which is classified as UVB. This is the UV light that is mainly responsible for causing sunburns and skin cancers. The highest energy containing UV light, UVC, is mostly absorbed by the ozone layer, but is the most effective at killing germs. An embodiment of this invention exposes the exhaled air to this germ killing UVC light, disinfecting it before releasing it back into the room air.

The primary purpose of this invention is the sanitization of the exhaled air prior to the nebulizer releasing it back into the environment from a nebulizer. An embodiment of this invention directs the exhaled air through a UVC light chamber to disinfect the air before releasing it back into the environment. UVC is commonly used as a method of disinfecting air and water, and studies have shown that UVC light can kill 99.9% of viruses in air, including the COVID-19 virus. Using UV light disinfection is not a new concept in itself, as Niels Finsen won the 1903 Nobel Prize for his discovery that UV light kills germs. This invention takes that concept and uniquely applies it to disinfect exhaled air exiting a jet nebulizer, making it, once again, safe for patients to use nebulizers in hospitals and other health care or crowded environments.

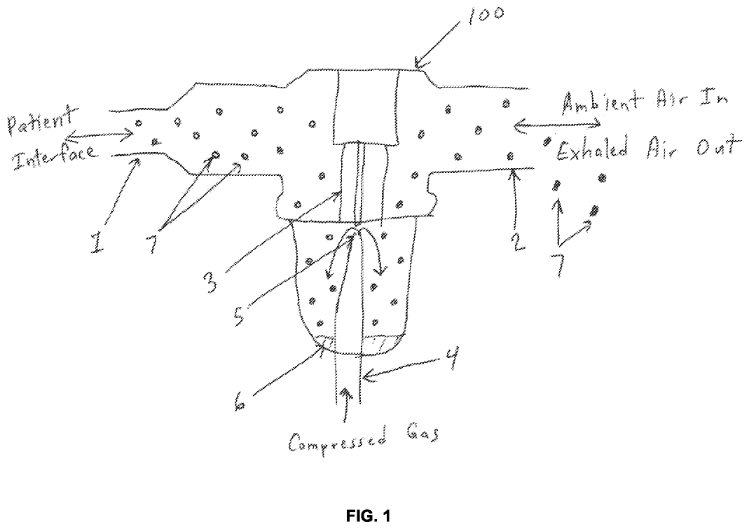

FIG. 1 illustrates a basic jet nebulizer 100. When the jet nebulizer 100 is in operation, there are three interfaces where something enters or exits the nebulizer: patient interface 1, ambient air interface 2, and compressed gas interface 4.

The most critical interface is the patient interface 1, which is what allows the patient to breathe through the jet nebulizer 100. For the purposes of this invention, the patient interface 1 is defined as anything that connects the mouth and or nose of the patient to the nebulizer 100, and can include tubing, mouthpiece, mask, or anything else that allows the patient to breathe in the aerosol medication in the jet nebulizer 100. The patient interface 1 is where the medicated air is inhaled by the patient from the jet nebulizer 100, and where the exhaled breath is pushed back into the jet nebulizer 100.

Ambient air interface 2 is where ambient air is pulled into jet nebulizer 100 and where exhaled air is allowed to escape jet nebulizer 100. Air is pulled into jet nebulizer 100 from the room environment when the patient inhales through patient interface 1. The ambient air enters jet nebulizer 100 via ambient air interface 2 and carries the atomized medication 7 to the patient via the patient interface 1. The medicine is drawn into the patient's lungs, and then the patient exhales air back into jet nebulizer 100 via the same patient interface 1. This exhaled air will carry some of the nebulized medication 7 as well as any contaminant dispersed in the exhaled air stream out of the nebulizer, exhausting it into the room environment via ambient air interface 2.

Compressed gas interface 4 is where compressed gas is introduced into jet nebulizer 100, which is used to create the atomized medication 7 that is delivered to the patient via patient interface 1. Liquid medication is transferred into cup 6, typically called a nebulizer cup or medicine cup. Cup 6 is typically removed from jet nebulizer 100 for medication to be transferred into it, and then cup 6 is reattached to jet nebulizer 100 for the patient to use.

Generally speaking, a jet nebulizer 100 causes the liquid medication to atomize by using compressed air entering jet nebulizer 100 via the compressed air interface 4 and allowing it to travel through a tube with decreasing diameter until it exits the tube via a small hole called the Venturi 5. At Venturi 5, the gas velocity increases as the gas pressure decreases, creating a vacuum that draws the liquid medicine up into small tubes. The small tubes deposit the liquid medicine in small droplets, or atomized medication 7.

Controlling droplet size in a nebulizer is critical in administering the atomized medication 7 to the patient, and the droplet size can be determined by a variety of factors. The most basic factors that impact atomized medication 7 droplet size are compressed air pressure and the size of the small tubes that initially create the droplets. Even with a constant compressed air pressure and tube size, the droplet size still varies due to the random nature of droplet creation. A jet nebulizer 100 includes baffles 3, which act to remove the larger droplets from the air inside the jet nebulizer 100. Large droplets will collide with baffles 3 and drop back into cup 6, while the smaller droplets will make it past baffles 3 and potentially be inhaled by the patient. The design of the baffles is critical to control the atomized medication droplet size.

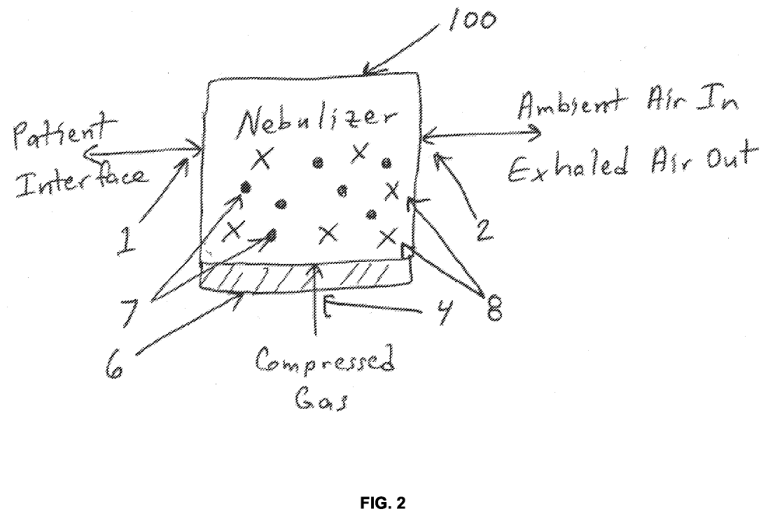

FIG. 2 illustrates a simplified material balance diagram of a jet nebulizer 100. The material balance view of the jet nebulizer 100 makes some of the flaws of nebulization easy to see. A material balance view focuses on the inputs and outputs of a process, with inputs and outputs being in balance in a closed environment. As discussed previously and shown in FIG. 2, the jet nebulizer has three interfaces where something enters or exits the nebulizer, the patient interface 1, the ambient air interface 2, and the compressed gas interface 4. The liquid shown in the bottom of cup 6 is part of the material balance, even though it is introduced into the jet nebulizer 100 before the process begins. The main difference between the processes illustrated by FIG. 1 and FIG. 2 is the addition of germs (or other contagions) 8, which are shown in jet nebulizer 100 along with the atomized medication 7.

As stated previously, the most critical interface is the patient interface 1, mainly because the purpose of the process is to transfer the atomized medication 7 to the lungs of the patient. From a material balance perspective, the patient breathes in air and atomized medication 7 through the patient interface 1, and exhales air and atomized medication 7 back into the patient interface 1. A portion of the atomized medication 7 is deposited in the lungs of the patient and does not return to jet nebulizer 100. In a perfect world, all of the medicine would be atomized and transferred so that all of the medicine would be deposited in the lungs of the patient. However, studies have shown that less than fifty percent of the medication finds its way to and remains in the lungs of the patient. Droplet size has a significant impact on drug delivery, as the droplets can be too large to reach the lungs of the patient, or too small to be deposited into the lungs. In both cases, medicine-laden droplets that are too large or too small will exit jet nebulizer 100 and enter the patient via the patient interface 1, but will have no impact on patient medication. The large droplets will remain in the patient outside of the lungs; the small droplets will simply be exhaled back into the jet nebulizer 100. This is why controlling the droplet size is so important to the nebulization process.

The compressed air interface 4 is not as critical to material balance because its function is simply to atomize the medicine. The compressed gas will exit jet nebulizer 100 by either being inhaled into the patient via patient interface 1, or by being pushed out of the jet nebulizer 100 via the ambient air interface 2 when the patient exhales.

The liquid medicine is introduced into the cup 6 before the process begins, and the liquid medicine will either be atomized and leave the nebulizer via patient interface 1 or ambient air interface 2, or the medicine will remain in liquid state to be removed when the cup 6 is disconnected for cleaning or re-supply. One of the major complaints of using jet nebulizers is that a portion of the atomized medicine 7 will always exit the jet nebulizer 100 via the ambient air interface 2 which means that that portion of the liquid medicine is not delivered to the patient. Over fifty percent of the atomized medication 7 can be released into the surrounding air by being forced out of the ambient air interface when the patient exhales.

The major difference between FIG. 1 and FIG. 2 is the introduction of germs 8 into the material balance. If the patient has germs that can be exhaled, like bacteria and viruses, the exhaled germs 8 will be exhaled into the jet nebulizer 100 via the patient interface 1. This does not show up in FIG. 1, because contagions or germs 8 are not intended to be introduced into the jet nebulizer 100. The COVID-19 pandemic changed the way the world has looked at contagions or germs 8, with mask wearing and social distancing becoming more the norm than the exception. With an airborne virus such as COVID-19, the likelihood of that virus being exhaled into the jet nebulizer 100 by an infected patient is high.

As with any other input material, any contagions or germs 8 introduced into the jet nebulizer 100 by the patient must escape the closed system. Because the contagions or germs 8 are introduced into the system via the patient interface 1, it is safe to assume that while some of the contagions or germs 8 will exit the jet nebulizer 100 via the same patient interface 1 during inhalation, at least a portion of the contagions or germs 8 will exit into the ambient air via the ambient air interface 2. This creates a concern that airborne contagions can be transmitted from an infected patient into the ambient air during the nebulization process, which has caused use of nebulizers to be reduced during the COVID-19 pandemic. The most basic concern is that the design of the jet nebulizer 100 does not take into account the patient introducing contagions into the closed system, which have no other designed exit path than to be released into the ambient air via the ambient air interface 2. This invention focuses on this limitation of the jet nebulizer 100, and provides a solution to handling and/or treating any contagions that are exhaled into the jet nebulizer 100 by the patient via patient interface 1.

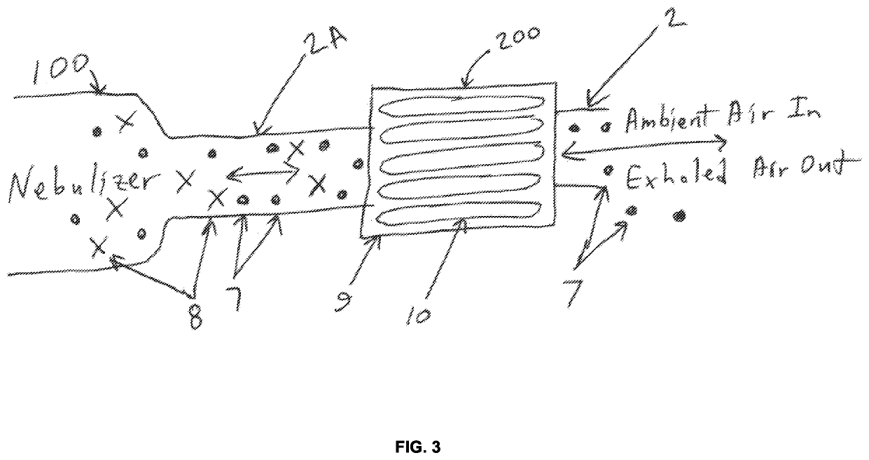

FIG. 3 illustrates an exemplary disinfecting chamber 9 placed on the ambient air interface 2 side of the jet nebulizer 100, between the jet nebulizer 100 and the ambient air interface 2. The embodiment shown in FIG. 3 directs the exhaled air through a UVC light disinfecting chamber 9 to disinfect the air, before releasing it back into the ambient air environment via the ambient air interface. The disinfecting chamber 9 is shown with multiple UVC light bulbs 10, which are placed around the tube where the exhaled gas exits the jet nebulizer 100. One major benefit of using UVC light in this application is that it produces no back pressure on the jet nebulizer 100, which would make it more difficult for the patient to breathe. UVC light is used to demonstrate the basic premise of this invention of disinfecting the exhaled air before releasing it into the ambient air environment via the ambient air interface, but one of ordinary skill in the art could use any suitable disinfecting method and all such methods and systems are within the scope of this invention.

UVC is commonly used as a method of disinfecting air and water, and studies have shown that UVC light can kill 99.9% of viruses in air, including the COVID-19 virus. UVC light is commonly used in healthcare settings to disinfect surfaces, equipment, and Personal Protective Equipment (PPE), which demonstrates confidence in UVC in killing germs like bacteria and viruses.

The high energy level of UVC light that allows it to kill germs also introduces potential risks with its use. It is important to ensure that the UVC light is concentrated on the potentially infected air, and that the patient is shielded from the UVC light. The disinfecting chamber 9 is constructed to focus the disinfecting method to the exhaled air. In one embodiment of this invention, the disinfecting method will be UVC bulbs 10, and the disinfecting chamber will be designed to both focus the UVC light onto the air, while also containing the UVC light within the chamber. Containing the UVC light is critical to the safety of the patient as well as others who may be in close proximity to the disinfecting chamber 9. The specific design and materials of construction for the disinfecting chamber 9 are specific to the disinfecting method being used, and in the case of UVC light, attention must be paid to preventing the UVC light from escaping the disinfecting chamber 9. Any design of a system using any UV light for disinfecting the exhaled air will need to ensure that the UV light source is properly shielded from the patient. Such shielding is known to those of ordinary skill in the art and any such shielding can be used in the present invention as a matter of design choice.

The ability of UVC light to kill germs or other contagions is dependent on the intensity of the UVC light, the wavelength of the UVC light, the ability of the UVC light to come into contact with the germs, and the amount of time that the germs are exposed to the UVC light. The optimum UV light wavelength can vary depending on the germs being targeted, while the intensity of the UVC light and time required to kill the germs has an inverse relationship. The more intense the UVC light is, the shorter time required to kill the germs, while a less intense UVC light will require a longer time to kill the germs. These variables are designed into the system on a case by case basis and in consideration of the particular germs being targeted. The disinfecting chamber may also be constructed in such a way as to impart a cyclone action to the air stream, decreasing the likelihood that germs could escape into the ambient air environment without having been exposed to the UVC light.

The disinfecting chamber 9 may be positioned between the jet nebulizer 100 and the ambient air interface 2 as shown in FIG. 3 in order to disinfect the exhaled air stream prior to exiting through ambient air interface 2. However, the location of the disinfecting chamber 9 in the nebulization system is not critical, as long as it is positioned to kill the germs 8 before they are released into the ambient air. In an embodiment, the disinfecting chamber 9 is located as close to the jet nebulizer 100 as possible, on the ambient air interface 2 side of the jet nebulizer 100. It is also possible to enclose the entire jet nebulizer 100 in a disinfecting chamber 9, to kill the germs while they are still in the jet nebulizer 100.

Another, but possibly less feasible, configuration would be to locate the disinfecting chamber 9 between the patient interface 1 and the jet nebulizer 100. This would reduce germs 9 before they reach jet nebulizer 100; however, the length of tubing required to provide enough time to kill germs would move the jet nebulizer 100 further from the patient interface 1, creating the potential for more of the atomized medicine 7 to coalesce and increase in size, thus reducing the efficiency of the medication process resulting in less medication actually reaching the lungs.

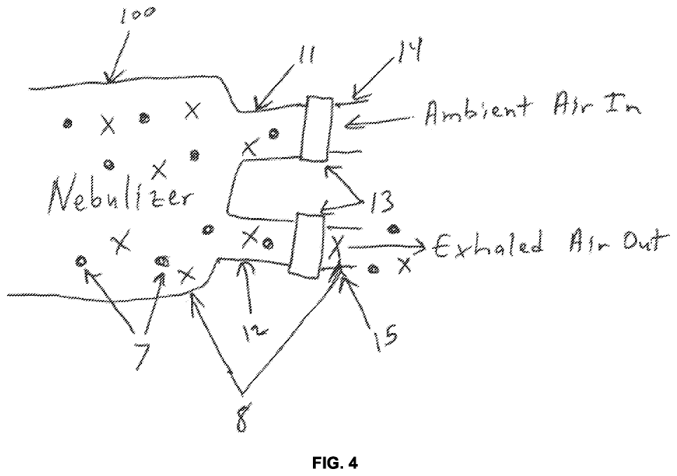

In certain embodiments, the disinfection process could be simplified by separating the ambient air infeed from the exhaled air outfeed. FIG. 4 illustrates one way in which the ambient air infeed 14 can be split from the exhaled air outfeed 15. In this embodiment, the jet nebulizer housing is modified to have two jet nebulizer feeds 11 and 12 on the ambient air side, not distinguishable from each other. One-way valves 13 are attached to the jet nebulizer feeds 11 and 12. The one-way valves 13 are designed to only allow air to flow in one direction through them, and are placed on the jet nebulizer feeds 11 and 12 in opposite flow orientations. One of the one-way valves 13 will only allow air to flow into the jet nebulizer 100 from the ambient air environment creating an ambient air infeed interface 14, while the other one-way valve 13 will only allow air to flow out of the jet nebulizer 100, creating an exhaled air outflow interface 15. This separation of the ambient air inflow from the exhaled air outflow allows the exhaled air to be diverted for disinfection or containment (not shown).

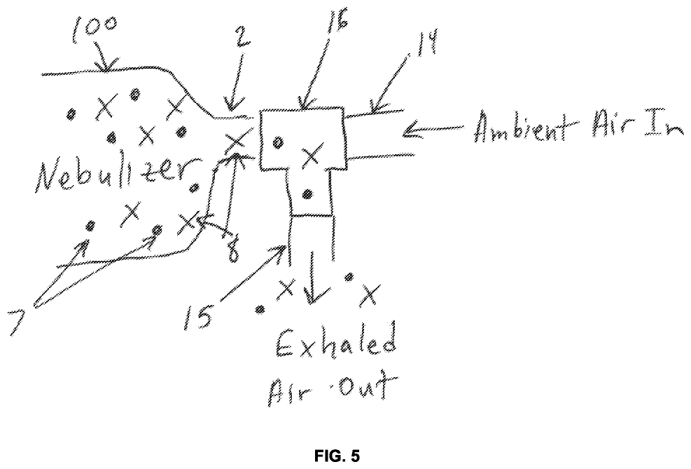

FIG. 5 shows another alternative embodiment having separate interfaces on the ambient air side. Separation is accomplished by using a T-shaped fitting on the ambient air interface of the jet nebulizer 100. Ambient air T-splitter 16 is shown attached to the ambient air interface 2 of a standard jet nebulizer 100 in FIG. 5. However, ambient air T-splitter 16 could be attached between the patient interface and the jet nebulizer. Ambient air T-splitter 16 controls the directional flow of air, so that air can only flow in through the ambient air infeed interface 14, and air can only flow out of the exhaled air outfeed interface 15. Ambient air T-splitter 16 uses one-way valves to control directional flow. An advantage of employing an ambient air T-splitter is that it can be attached to a standard jet nebulizer, while the nebulizer design shown in FIG. 4 would require modifications to a standard jet nebulizer.

The purpose of splitting the single ambient air interface into an ambient air infeed interface and an exhaled air outfeed interface is to ease the process of disinfecting or containing the exhaled air. This split interface ambient air T-splitter provides the most flexibility in choosing disinfection options. Because patients taking nebulizing breathing patients commonly have difficulty breathing, it is desired to keep the path from ambient air interface to the patient interface as short as possible. Splitting the ambient air infeed interface from the ambient air outfeed interface with a T-splitter allows the ambient air infeed interface to remain the same length from the patient interface as in current nebulizer designs, while the ambient air outfeed interface can be lengthened or rerouted to allow disinfection without impacting the patient's breathing.

All references cited in this specification, including without limitation, all papers, publications, patents, patent applications, provisional patent applications, presentations, texts, reports, manuscripts, brochures, books, internet postings, journal articles, and/or periodicals are hereby incorporated by reference into this specification in their entireties, including all figures and tables, to the extent they are not inconsistent with the explicit teachings of this specification. The discussion of the references herein is intended merely to summarize the assertions made by their authors and no admission is made that any reference constitutes prior art. Applicants reserve the right to challenge the accuracy and pertinence of the cited references.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the versions contained therein.

What is claimed is:

1. In a nebulizer having a nebulizer housing, a patient interface, an ambient air infeed for carrying inhaled air to the nebulizer for entraining a medication for administration to a patient's lungs, and an exhaled air outfeed for carrying exhaled air from the lungs through the exhaled air outfeed to be treated or contained where the exhaled air may carry contagions entrained therein and could enter the contagions into the environment if not prevented, the improvement comprising a disinfecting chamber attached to the nebulizer for reducing the amount of contagions entering the environment from the exhaled air outfeed.

2. The nebulizer of claim 1 wherein the disinfecting chamber comprises a UVC light source capable of reducing the amount of contagions.

3. The nebulizer of claim 1 wherein the disinfecting chamber is designed to create a cyclone flow pattern for exhaled air flowing through the disinfecting chamber.

4. The nebulizer of claim 1 wherein the disinfecting chamber is removably attached to the nebulizer housing.

5. The nebulizer of claim 1 wherein the disinfecting chamber comprises a filter capable of reducing the amount of contagions.

6. The nebulizer of claim 1 further comprising a device that separates inhaled air from exhaled air.

7. The nebulizer of claim 6 wherein the device is removably attachable to the nebulizer housing.

8. The nebulizer of claim 6 wherein the device is a T-splitter.

9. The nebulizer of claim 6 wherein the device comprises at least two one-way valves.

10. The nebulizer of claim 6 wherein the device is positioned between the patient interface and the nebulizer.

11. The nebulizer of claim 6 wherein the device is positioned for communication with the exhaled air outfeed.

12. The nebulizer of claim 6 wherein the device is attached to the exhaled air outfeed.

13. A nebulizer comprising:
a) a nebulizer housing;
b) a patient interface;
c) an ambient air infeed for carrying inhaled air to the nebulizer for entraining a medication for administration to a patient's lungs
d) an exhaled air outfeed for carrying exhaled air from the lungs through the exhaled air outfeed to be treated or contained where the exhaled air may carry contagions entrained therein and could enter the contagions into the environment if not prevented; and
e) a disinfecting chamber attached to the nebulizer for reducing the amount of contagions entering the environment from the exhaled air outfeed.

14. A nebulizer comprising:
a) a nebulizer housing;
b) a patient interface;
c) an ambient air infeed for carrying inhaled air to the nebulizer for entraining a medication for administration to a patient's lungs;
d) an exhaled air outfeed for carrying exhaled air from the lungs through the exhaled air outfeed to be treated or contained where the exhaled air may carry contagions entrained therein and could enter the contagions into the environment if not prevented; and
e) a device that separates inhaled air from exhaled air.

15. The nebulizer of claim 14 wherein the device is a T-splitter.

16. The nebulizer of claim 14 wherein the device comprises at least two one-way valves.

17. The nebulizer of claim 14 wherein the device is positioned between the patient interface and the nebulizer.

18. The nebulizer of claim 14 wherein the device is positioned for communication with the exhaled air outfeed.

19. The nebulizer of claim 14 wherein the device is attached to the exhaled air outfeed.

20. A device for removably attaching to a nebulizer that has nebulizer housing, a patient interface, an ambient air infeed for carrying inhaled air to the nebulizer for entraining a medication for administration to a patient's lungs, an exhaled air outfeed for carrying exhaled air from the lungs through the exhaled air outfeed to be treated or contained where the exhaled air may carry contagions entrained therein and could enter the contagions into the environment if not prevented, wherein the device is a T-splitter comprising at least two one-way valves and is adapted for positioning between the patient interface and the nebulizer, for communication with the exhaled air outfeed, and for attachment to the exhaled air outfeed.
